(19) United States
(12) Patent Application Publication
(10) Pub. No.: US 2014/0180065 A1
(43) Pub. Date: Jun. 26, 2014

(54) FIDUCIARY MARKERS AND METHODS OF PLACEMENT

(71) Applicant: The Regents of the University of California, Oakland, CA (US)

(72) Inventor: Maurice Garcia, San Francisco, CA (US)

(73) Assignee: The Regents of the University of California, Oakland, CA (US)

(21) Appl. No.: 14/116,735

(22) PCT Filed: May 10, 2012
(86) PCT No.: PCT/US12/37379

§ 371 Date: Nov. 8, 2013

Related U.S. Application Data
(60) Provisional application No. 61/518,742, filed on May 11, 2011.

(51) Int. Cl. A61B 19/00; A61B 6/00; A61B 6/12; A61N 5/10; A61K 49/04

(52) U.S. Cl. CPC A61B 19/54; A61N 5/1007; A61N 5/1014; A61K 49/04; A61B 6/12; A61B 6/481; A61B 6/485
USPC 600/424; 600/431; 600/6

(57) ABSTRACT

The invention relates to the field of radiation oncology, specifically the use of novel radio-opaque fiduciary markers which resist migration within tissues, which may be implanted in the body and imaged during radio-therapy to insure accurate treatment of target regions while avoiding irradiation of non-target regions. In one embodiment, the markers comprise oblong bodies from which a plurality of short tines protrude. Also provided are novel devices for implanting such markers. Additionally, the invention provides methods of delineating tumor resection beds and whole-bladder contours in the radiotherapeutic treatment of bladder cancer. Lastly, the invention encompasses novel methods of delivering fiduciary markers and other implants and materials by needle with sealing aids that increase the retention rate of the delivered markers, implants, or materials.

### CROSS-REFERENCE TO RELATED APPLICATIONS

This application is based on and claims the benefit of priority of U.S. Provisional Patent Application Ser. No. 61/518,742, filed on May 11, 2011, the contents of which are incorporated by reference in their entirety, and PCT Application Serial Number PCT/US2012/037379, filed on May 10, 2012, the contents of which are incorporated by reference in their entirety.

### STATEMENT REGARDING FEDERALLY SPONSORED RESEARCH OR DEVELOPMENT

This invention was made with United States government support under Grant Number 5-K08-HD069462-02 awarded by the National Institutes of Health. The United States government has certain rights in the invention.

### REFERENCE TO SEQUENCE LISTING, A TABLE, OR A COMPUTER PROGRAM LISTING COMPACT DISK APPENDIX

Not Applicable

### FIELD OF THE INVENTION

The invention relates to the field of radiation oncology, specifically the use of novel radiopaque fiduciary markers which resist migration, devices for implanting them, and general methods for improving the retention of materials injected into the tissues of the body. Additionally, the invention comprises novel schemes for improving the targeting of therapeutic radiation doses in the treatment of bladder cancer.

### BACKGROUND OF THE INVENTION

External radiation therapy is used to treat a variety of cancers and other conditions. In most forms of external radiation therapy, a radiation beam is directed at a target internal to the body. Accurate targeting of radiation is complicated by the fact that the location of various tissues within the body is not static. This is especially true of target areas within the lower abdomen, where organs and tissues are constantly changing shape, size, and location due to the movement of digested food through the digestive tract or the retention and expulsion of urine in the bladder. Additionally, although it is attempted to keep patients in consistent poses in each treatment session, it is difficult to identically position a patient from session to session. Accordingly, small radiation targets within the tissues and organs of this region do not remain in a static location.

In most forms of radiotherapy, the radiation delivery mechanism is integrated with an imaging modality, usually conebeam CT or portal imaging. Unfortunately, these imaging modalities do not resolve soft tissues well. Therefore, a radiation oncologist aiming a radiation source at a target area is often unable to directly view and locate the target area at the time of actual treatment. Instead, the location of target areas is estimated, based upon previous imaging of the target area, for example by MRI or CT scan, which is then mapped to external landmarks on the body or bony landmarks within the body. As a result, the targeting of radiation is often inaccurate. In order to increase the probability of successfully treating the target area, the treatment area is often enlarged to encompass the probable region of the target area, resulting in irradiation of healthy non-target areas (overtreatment) and a risk of missing the intended target area (undertreatment).

The treatment of bladder cancer exemplifies the difficulties of accurately targeting radiation to soft tissue areas in the body. One treatment regimen for muscle invasive bladder cancer is the resection of the tumor from the bladder wall, followed by radiation treatment. The radiation treatment is commonly administered in more than one dose, for example, based upon the pre-treatment dose-area planning CT scan, the target area may be sub-divided into field areas, which are treated with varying doses of radiation. A high-field dose is directed at the site of the tumor resection bed, and a lower-field dose is directed at the entire bladder. Unfortunately, the bladder changes shape, size, and 3-D position throughout the day from being filled and emptied of urine and due to its proximity to the bowels, which are in a constant state of movement. Direct imaging of the tumor resection bed and the contours of the whole bladder are not possible with conebeam CT or portal imaging. Therefore, the high-field and low-field doses are directed at regions estimated to contain the resection bed and whole bladder, respectively, based upon past locations of these target areas in previous direct imaging and mapping of these snapshot locations to external body landmarks or bony landmarks within the body.

As a solution to the general problem of resolving soft tissue target areas, it is known in the art that radiopaque fiduciary markers may be implanted within or around the target area. These radiopaque markers may be visualized on conebeam CT or portal imaging devices and allow the Radiation Oncologist to direct the therapeutic radiation more precisely to the target area. For example, in treatment of prostate cancer, the placement of small gold fiduciary markers is practiced, for example as described in Reference 1. In the treatment of bladder cancer, the use of gold fiduciary markers has been tested, as described in Reference 2.

While the use of radiopaque fiduciary markers is promising, there are potential complications that result from the migration of the markers out of the area into which they have been placed. For example, gold markers placed in the prostate have been found to migrate out of the position, and have even been reported to enter the circulatory system, for example, as reported in Reference 3. Likewise, in the preliminary study of gold fiduciary markers in the bladder, 25% of the markers fell out of place, as reported in Reference 2. Similar migration problems have been reported with other types of implants. For example, in interstitial brachytherapy, small radioactive rods are placed within or around cancerous areas by needle injection. It is widely reported that such implanted objects can migrate out of the tissue they have been placed into, for example as reported in Reference 4 and Reference 5.

Markers designed to resist migration are known. For example, described in U.S. Pat. No. 5,941,890 (to Voegle et al., issued Aug. 24, 1999) and U.S. Pat. No. 6,425,903 (to Voegle, issued Jul. 30, 2002) are barbed, “W” shaped surgical markers having camming action. In another example, very recently, a new product comprising a ribbed gold fiduciary marker with a scored surface has been introduce on the market (Surelock™ markers by CIVCO). However, it is as yet unknown whether the ribbed design and scoring on these new markers will overcome the propensity of implanted markers to migrate.

Accordingly, there is a need in the art for solutions which prevent the migration of implanted fiduciary markers and other implanted objects from their site of introduction. In the treatment of bladder cancer, for example, there is a need for improved targeting of high-radiation doses to tumor resection beds and improved accuracy in targeting low-field radiation doses to the whole bladder.

The inventions disclosed herein address these unmet needs and provide the art with various devices and methods for accurately locating target areas within the body.

### SUMMARY OF THE INVENTION

In one aspect, the invention provides novel fiduciary markers that are resistant to migration from their site of implantation and which may be used to guide radio-therapeutic treatment. In another aspect, the invention provides novel delivery systems for such fiduciary markers. In another aspect, the invention provides methods of implanting markers or other materials in the body with improved probability of retention. In another aspect, the invention provides kits comprising novel markers, retention aids, and delivery devices.

### DESCRIPTION OF THE DRAWINGS

FIG. 1A depicts a marker of the invention comprising a body (101) of radiopaque material, from which four rows of anchoring moieties (102 and 103) protrude, each row oriented 90 degrees from each other. The anchoring moieties on one half of the body (102) have an orientation opposite of that of the anchoring moieties on the other half of the body (103). FIG. 1B depicts a coaxial needle for the delivery of the markers. The coaxial needle comprises a hollow barrel (105) with a beveled point (108). At the distal end of the hollow barrel is a lip or handle which may be gripped with the fingers (109). Within the hollow tube is a solid stylet (106) capable of movement within the outer sheath. At the distal end of the stylet is affixed a pad or plate (107) which may be pressed to move the stylet within the barrel. Loaded within the tip end of the barrel is a plug of packing material (110). Distal to the packing material is a marker (111). Distal to the marker is a plug of sealing material (104).

FIG. 2 depicts an anterior view of the bladder. A coaxial placement needle (203) is inserted into the bladder through the urethra and used to implant markers at various locations. Markers (204) are placed around a tumor resection bed (202) to delineate its position. Anatomic markers (205) are also placed at various places, marking the anatomic boundaries of the bladder.

FIG. 3 depicts a cross section of the bladder. The bladder muscle layer (301) underlies the bladder mucosa layer (302). A coaxial placement needle (303) is inserted transversely into the bladder mucosa layer and deposits a marker (304) and a plug of sealing material (305).

FIG. 4 depicts a cross section of the bladder. The bladder muscle layer (401) underlies the bladder mucosa layer (402). The tumor resection bed (403) is an area where cancerous bladder mucosa tissue was previously removed. Markers (404) are placed within the bladder mucosa layer at a short distance from the margin of the tumor resection bed.

FIG. 5 depicts a tumor resection bed (501) around which three markers (502, 503, and 505) have been placed in a staggered orientation. The staggered orientation insures that each of the three markers will be distinctly visible (i.e. will not occlude visualization of the other markers) when viewed from either the anteroposterior or lateral viewpoint.

### DETAILED DESCRIPTION OF THE INVENTION

Radio-Opaque Fiduciary Markers

As used herein, the term “marker” will refer to a radio-opaque fiduciary marker that is placed within the body in a specific location to mark the location of a feature, for example a tumor site or the boundaries of an organ or tissue.

The markers of the invention comprise a body. The body of the marker may be of any shape, for example cylindrical, cigar shaped, disc shaped, star shaped, square shaped, curved, spherical, etc. The body of the marker optionally may comprise one or more holes, which may aid in the identification of the marker, or which may be used to aid in suturing or stapling the marker in place. A preferred body shape is oblong, for example that of an elongated cylinder, cigar shaped, or other shapes with a length of 150%, 200%, 300% or greater in size than its width. Such oblong shapes are amenable to needle delivery.

The size of the marker may range from 0.1 mm² to a maximum size of several square centimeters, for example 2, 4, 8, or 20 cm², depending on where in the body the marker is placed. At a minimum, the size of the marker must be within the visualization resolution of the radiographic imaging modality that will be used in conjunction with the maker. For example, if the markers are to be imaged using a low-resolution modality, a larger sized marker will be required than would be employed for a high-resolution imaging method. In general, the use of the smallest sized marker that may be easily visualized in a selected tissue type or organ with a selected mode of radiographic imaging is preferred. This is because a smaller marker will reduce the amount of trauma and the risk of infection caused by the placement of the marker and its presence in the tissue. However, markers smaller than about 0.25 mm in length and width are generally not preferred, as they risk being taken up in the circulatory system, which could cause adverse consequences.

Exemplary markers include cylindrical markers ranging from 0.5 to 3 mm in length and from 0.5 to 1.1 mm in width. An exemplary marker that may be used in the bladder is a cylindrical marker with a body size of 1.5 mm in length by 1 mm wide. Such a marker will be readily resolved on most imaging modalities, for example X-ray, CT, MRI, fluoroscopy, and others.

The composition of the marker body is not limited to any specific material, so long as the marker body, as a whole, is adequately radio-opaque to be visualized by an imaging modality selected for visualization of the marker. “Radio-opaque,” or “radiopaque,” as used herein refers to the property of inhibiting the passage of electromagnetic radiation. Exemplary radio-opaque materials include gold, silver, copper, palladium, platinum, and alloys thereof. Preferred materials are those, such as 24 karat gold, which are highly isodense, and consequently are very radiopaque, and which also do not elicit an immune response from the body. The composition of the marker body may be of a single material, or may comprise layers or composites of different materials. For example, the body may comprise a gold alloy which is coated in a thin layer of 24 karat gold. Alternatively, the body may comprise conglomerate material with radio-opaque materials embedded in a non-radio-opaque matrix, such as an organic polymer material.

In some embodiments, the marker body comprises discreet regions of radio-opaque and radiolucent materials. For example, radio-opaque bodies (e.g. spheres or cubes) may be encapsulated at fixed distances from each other within a radiolucent material, for example Endolign™ (Ivibio) carbon polymers. The resulting body will appear on radiographic imaging equipment as multiple discreet bodies. In this way, distinct markers can be coded and distinguished from one another. For example, bar coding may be accomplished by including radiopaque materials of varying widths, at varying distances.

The markers of the invention further comprise one or more anchoring moieties. The function of the anchoring moiety is to prevent the slippage and movement of the marker within the tissue into which it is placed. The anchoring moiety comprises a projection from the plane of the marker body which physically embeds the marker within the tissue, both in the short term, and long term time scales. Immediately following implantation, the anchoring moiety provides resistance to migration by burrowing or hooking into surrounding tissue. Typically, the implantation of the marker will create a localized wound response, and both scar and healthy normal tissue will form around the marker. When one or more anchoring moieties are employed, over time, these projections are engulfed in scar and healthy normal tissue and the body of the marker is held in place, resisting longitudinal or rotational movement. By creating an irregular surface with interstices, and by creating surface area that is oriented in multiple dimensions, the anchoring moieties provide an irregular scaffold for scar tissue growth that will immobilize the marker body in place.

Preferably, multiple anchoring moieties are employed on each marker, as this will aid in anchoring the marker while reducing the necessary size of the anchoring moieties. Multiple anchoring moieties arrayed in rows, for example, will create an irregular surface which is more likely to become embedded in scar tissue and which will resist movement.

The anchoring moiety may take on any shape. For example, the moieties may comprise triangular or pyramidal teeth, spines, tines, rods, or any other shape. Anchoring moieties having tapered or pointed ends are preferred. Alternatively, forked, barbed, branched, or curved projections having a hook-like structure may be utilized. Additionally, the anchoring moiety may comprise one or more holes or interstices, for example, a line of small holes through the body oriented along the long axis of the body, such holes providing a tunnel or depression into which scar tissue may grow and which serve to anchor the moiety in place.

The length and width of the anchoring moiety may be of diverse dimensions. Preferred anchoring moieties range from 0.01 mm to 1.0 mm in length, for example, from 0.05 to 0.3 mm in length.

The anchoring moiety may project from the body of the marker at any angle, ranging from, for example, almost flush with the plane of the marker body, to perpendicular. Preferred angles are in the range of thirty to sixty degrees. In most cases, it is preferred that multiple anchoring moieties will employed and that they will be angled in opposing directions, in order to resist movement in any one dimension.

The anchoring moieties of the invention may be arrayed in any configuration. For example, the anchoring moieties may be arrayed in one or more longitudinal rows along the axis of the marker body. The anchoring moieties may be configured at any desired density, for example 1-3 anchoring moieties per mm.

A preferred form of anchoring moiety is a tine. A tine, as used herein, refers to a prong, a spine, or a fang tooth-like projection. Exemplary tine shapes include both regular and irregular shapes resembling triangular prisms, pyramids, cones, and slender rods. Tines are preferably tipped with a sharp or tapered point. For a marker of ˜3 mm in length and 1 mm width, the preferred length of the tine is 0.05 to 0.5 mm, and generally such tines will be of relatively small size compared to the size of the marker body. Preferably, at least 4-12 tines are present on each marker. A marker of 2-3 mm in length will preferably have in the range of 24-30 tines, It is preferred that the tines be angled away from the plane of the body of the marker, for example at an angle of 20-60 degrees. Among the tines on a marker, it is preferred that a fraction of them be configured such that they are angled in one direction, and that the remainder of them be angled in a different direction, for example, in an opposing direction. Alternatively, various fractions of the tines may be oriented in multiple different directions. An advantage of using multiple, short tines is that they provide a great deal of anchoring while minimally increasing the effective footprint, e.g. diameter, of the marker body, and thus minimizing the trauma and risk of infection associated with implanting a marker.

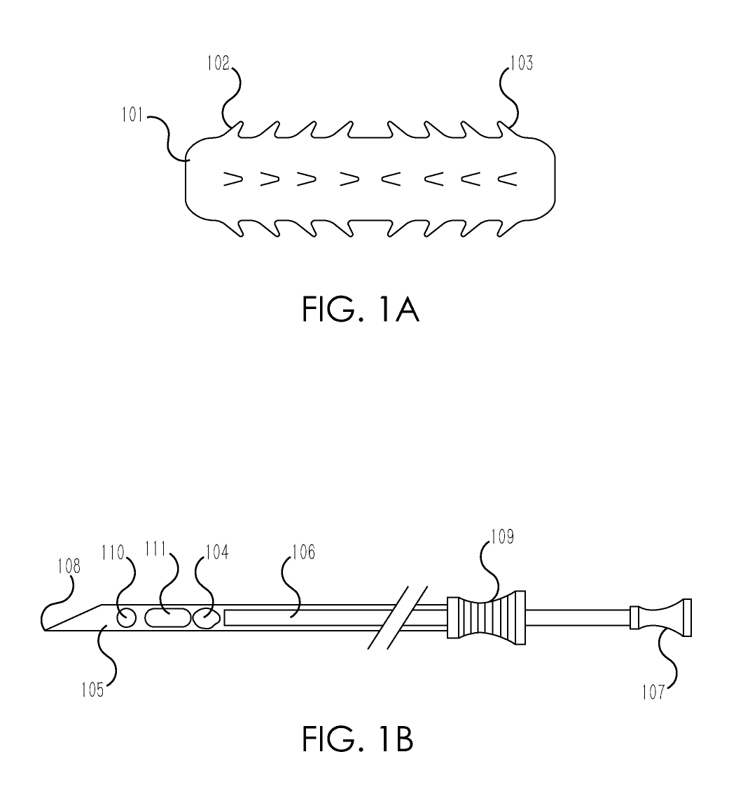

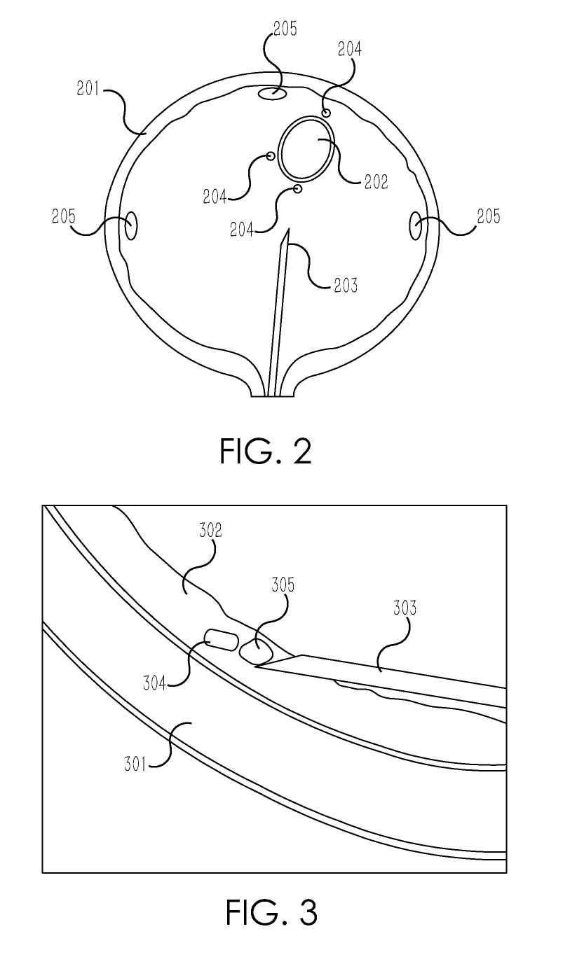

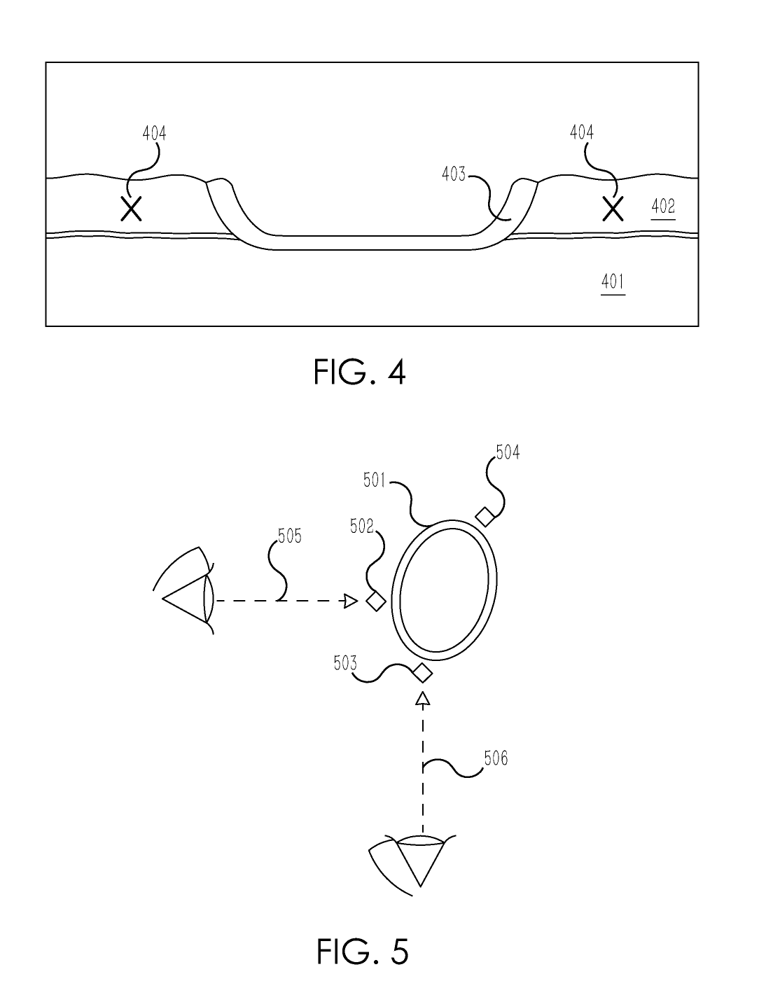

An exemplary embodiment, suitable for implantation in the bladder mucosa, comprises an elongated body of about 2-3 mm in length and about 0.5 to 1.25 mm in width. Four rows of anchoring moieties comprising tines having a triangular “tooth” like shape are placed along the long axis of a cylindrical marker body, oriented at 90 degrees from each other around the circular body of the marker, at a density of 2-3 anchoring moieties per mm. The anchoring moieties are angled at 45 degrees from the body of the marker. Bisecting the long axis of the marker, the markers on the opposing halves are angled in opposite directions, as depicted in FIG. 1.

The anchoring moiety may be of any composition. The function of the anchoring moiety is simply to anchor the marker, and it is not necessary that the anchoring moiety be radio-opaque. Preferred compositions for the anchoring moiety include any material that does not elicit an immune response, for example, 24 karat gold.

The markers of the invention may be manufactured by any means, including lathing, drilling, engraving, milling, etching, welding and other well known fabrication techniques appropriate for the selected materials and the scale of the markers. In some embodiments, the anchoring moieties are created separately from the body of the marker, and are then attached to the body. For example, micro-welding or wire bonding techniques, as known in the art, may be used to attach anchoring moieties to the marker body. In some embodiments, the anchoring moieties are created by modifying the body material. For example, tooth-like anchoring moieties can be created on a 24K gold wire (for example 17 AWG) by slightly cutting into the wire at an angle with a sharp blade, which peels or chips a tooth-like flap from the body of the wire.

Optionally, the markers may be coated with various materials that perform ancillary functions. For example, insertion of the markers into a tissue will generally cause localized trauma to that area. The markers may be coated with compounds known in the art such as antibiotics, anti-infectives, anti-inflammatory substances, and substances that promote wound healing, such as insulin, growth factors, or hemostatic agents. In those instances where a marker is being used to delineate a tumor site in order to aid in a radiation therapy regimen, it may be advantageous to coat the markers with radio-sensitizing substances known in the art, for example metronidazole or misonidazole, which would be expected to increase the efficacy of the radiation treatment. Alternatively, when delineating a tumor site, the markers may be coated with chemotherapeutic agents known in the art, for example some of which (e.g. Cisplatin, Gemcitabine) have dual properties: they are both cytotoxic to cells of some tumors, and, are radiosensitizing—making the target area tissues more susceptible to radiation therapy. Some chemotherapeutic drugs also have the local effect of embolizing the blood vessels of some hypervascular tumors. Marker coatings may be formulated using any method known in the art, for example, spray drying of agents on the markers, dipping markers in solutions of agents, and other encapsulation techniques. Any number of inert carriers or delivery vehicles known in the art may be employed in order to tailor the release kinetics of the ancillary agents to their desired function.

To facilitate tracking of the markers in real-time, the markers may be composed of different materials, so that specific markers can be individually tracked by imaging and/or a radio-frequency interface, in real-time. For example, when the isodenisty of different markers can be discerned by a particular imaging modality, or, when the electric charge of different markers can be detected to distinguish among different markers, all of the markers placed into a patient can be distinguished from one another, and their location can be tracked, in real time. When the location of individual markers can be tracked in real-time, then, it is possible to construct a “spot” 3-dimensional image of the bladder based on the location of a limited number of markers.

Devices for Implanting the Markers

The markers of the invention may be placed into the body by any means. For example, the target organs or tissues may be accessed by open surgical methods and the markers placed into the target area by stapling, suturing, injecting, etc. Alternatively, the markers may be placed in the body by the use of endoscopic surgical instruments.

In a preferred embodiment, the markers of the invention are placed into the target organ or tissue by use of a coaxial needle. The coaxial needle consists of a hollow external tube and an internal stylet which has a diameter close to that of the outer tube, the stylet being moveable within the lumen of the outer tube. At one end of the needle (the “tip end”), the end of the stylet is withdrawn into the outer tube a small distance, allowing the tip of the outer tube to be loaded with a marker and other objects. When the stylet is pushed at the opposite end (“the distal end”) end, it will displace (push out) the contents of the needle lumen through the tip end of the outer tube. In some embodiments, the distal end is circumscribed by an attached handle, from which the distal end of the stylet protrudes. The end of the stylet may be fitted with a plunger or other substantially flat pad. The user grips the handle at the end of the needle with their fingers and then may depress the stylet with their thumb, causing the entire stylet to move towards the tip end and expelling the contents of outer tube lumen out the tip end. Alternatively, the user end of the stylet may be attached to a motorized pusher, which upon activation will move it towards the tip by a pre-set amount.

The end of the needle's outer tube is preferably tapered, which creates a sharp point that aids in its insertion into the target tissue, causing less local trauma than a blunt needle. The preferred angle of taper is about 30 to 50 degrees.

The needle may be of any diameter. Preferably, the needle diameter does not significantly exceed the diameter of the marker which it is delivering. The marker must be able to move within the needle, but markers may fall out of an excessively large needle. Additionally, larger than necessary needles will cause unnecessary trauma to the injection site.

The needle and internal stylet may be made out of any material. Preferred materials for the needle portion of the co-axial needle include stainless steel and other biocompatible metal alloys used in the manufacture of medical devices. In some embodiments, the co-axial needles of the invention will be deployed along the axis of an endoscopic instrument, such as a cystoscope, laparoscope, etc. In such cases, it is preferred that the coaxial needle be somewhat flexible to aid in the insertion and movement of the instrument in the body. In other embodiments, the placement of the needle will be aided by the use of radiographic imaging methods. Accordingly, in such cases it is preferred that the tip of the coaxial needle or some portion of it be radio-opaque so that the position of the needle tip may be readily imaged. For example, the outer portion of the needle may be made with materials having high radiopacity, or may be adorned or coated with such materials. In those embodiments where the coaxial needle is to be used in conjunction with endoscopic instruments, the length of the needle must be substantially the same length as the instrument. For example, when using a coaxial needle with a cystoscope for placement of markers in the bladder, a length of at least 32-35 cm is preferred, such that the coaxial needle may be used with standard cystoscopes, where ˜31 cm. is the minimum distance between the entry point of the working port through which the needle is advanced, and when the needle tip can be viewed by the scope's camera lens.

In some embodiments, it is preferred that the tip of the needle be curved, for example 1-3 cm of the needle at the tip which is bent, bowed, or otherwise angled anywhere from 5-90 degrees from the axis of needle. In many embodiments, the needle will be used in conjunction with an endoscopic visualization instrument, such as a cystoscope. The use of a curved needles allows the markers to be inserted into tissues laterally from the plane of the endoscopic visualization instrument. This is advantageous because it allows for the precise placement of the markers into thin layers of tissue, for example, the bladder mucosa or submucosal space. Additionally, the use of a curved needle allows insertion of the markers into difficult-to-reach areas. In such cases, it will be necessary that the outer sheath of the coaxial needle be made of a flexible “memory” material which may be straightened when passed through the rigid endoscopic instrument and which then springs back to its curved shape when extruded from the end of the endoscopic instrument. In such cases, it is necessary that the stylet, or at least the portion of the stylet extending into the angled portion of the coaxial needle, be flexible and able to move within the curved portion of the outer sheath. Exemplary memory materials include titanium and titanium alloys.

Sealing Materials

When a material is delivered into a target tissue by needle, the entry wound created by the needle creates a potential escape path for the implanted material. Advantageously, the inventor of the present disclosure has developed a method of promoting sealing of the needle entry wound to prevent the diffusion, migration, expulsion, or other removal of the delivered material out of the needle wound. This method, and associated compositions, may be used in various contexts, including the delivery of the novel markers disclosed herein, the delivery of other fiduciary markers known in the art, the delivery of therapeutic agents, or the delivery of medical implants of any kind.

In order to seal the needle entry wound, a small amount of sealing material is introduced behind (i.e., distal to) the implanted material. The sealing material acts, by various means, to physically block the wound and/or to induce physiological processes which seal the entry wound. For example, the sealing material may comprise a hemostatic agent, i.e. an agent that promotes vasoconstriction or clotting. Various hemostatic agents are known in the art, for example, microfibrillar collagen hemostats, chitosan hemostats, anhydrous aluminum sulfate, potassium alum and titanium dioxide. Alternatively, the sealing material may work by physically sealing the entry wound of the needle. Swellable materials which increase their size and bulk when wetted make excellent sealing materials. Swellable hydrogels and polymers are known in the art, including, for example poly(ethylene oxide). Swelling materials and hemostatic agents may be advantageously combined for increased efficacy. The sealing material is biocompatible, and is preferably bioabsorbable, so that it is not permanently retained in the body.

The bioabsorbable sealant described above can be impregnated with antibiotic materials, as known in the art, if desired, to further lower risk of infection.

An exemplary sealing material is Gelfoam™ (Pfizer) compressed sponge. Gelfoam is a well studied, porcine-derived gelatin compressed sponge material which swells to larger size upon absorbing body fluids, has hemostatic properties, and which is well tolerated in tissues, being absorbed after a period of days to weeks. Other exemplary sealing materials include Otopore™ (Polyganics), polyvinyl alcohol sponge materials (for example, Merocel™ (Beaver Vistec), oxidized regenerated cellulose (e.g. Surgicel™ (Ethicon 360) or Oxycel™ (Oxycel)) and collagen sponges (for example, Avitene™ (Bard Davol Inc.)).

The sealing materials of the invention may be introduced into the wound site in various forms. In a preferred embodiment, the sealing material is formed into a cylindrical plug by punching sheets of the material with a die of the same or substantially the same diameter as the needle which will be used to inject the plug into the wound site. This results in discs or cylinders of plug material. The preferred width of such plugs, in the dry state, is in the range of 75-99% of the lumen diameter of the delivery needle, allowing the plugs to move freely within the coaxial needle. The preferred length of such plugs is in the range of 10-200% of the length of the marker, implant, or other item being implanted. For example, when using a gold fiduciary marker of 2 mm in length, a plug of 0.2 to 4 mm in length is preferred.

Loading of Markers into Needles

The markers of the invention may be loaded into delivery coaxial needles in various configurations. For example, the markers may be held in place within the needle by plugging the needle tip with a packing material. The packing material may be any biocompatible substance which acts to physically hold the marker within the needle lumen and prevent its escape from the needle tip until the time of implantation. The packing material may be adherent, or may hold the needle in place by simple tension forces created by compressing the packing material into the needle. The packing material must be readily expelled from the needle and not block the path of the marker. Exemplary packing materials are powders, granules, waxes, pastes, and other solids or semi-solids.

In some embodiments, the packing material is inert and dissolvable in bodily fluids, and will quickly disperse when introduced into the target tissue. In other embodiments, the packing material performs a secondary function, or is intermixed with agents that perform secondary functions, such as anti-infectives, healing promoters, therapeutic agents, etc.

Multiple markers may be loaded into a single needle. For example, as discussed below, in some cases it will be advantageous to introduce a string of multiple markers in a single site in order to create a feature which is visually distinct. For example, a marker doublet or triplet may be easily distinguished from a single marker. In such embodiments, the markers may be loaded end-to-end adjacent to each other, or may be separated from each other by a plug of packing material in order to cushion the markers from each other or to create the desired spacing between them. Sealing materials may also be placed between the markers in order to prevent their clumping together and to maintain desired spacing. Markers may also be connected with a bioabsorbable material, for example bioabsorbable string, such that they are initially deployed in connected strands.

Markers, and sealing materials if used, may be loaded into the coaxial needle in a series of discreet units, allowing a singe needle to be used to inject multiple markers at multiple sites. This obviates the need to remove and replace the coaxial needle after each marker insertion. In such “trains” of multiple markers, the repeating unit consists of the following, arranged in order, moving from the tip towards the distal end of the lumen: a marker (or multiple markers if a “string” is to be used), a body of sealing material (optional), and a plug of packing material that acts as a spacer between units. The user may then inject a marker or markers (and a plug of sealing material, if used) at a site, relocate the needle to the next site, and inject another marker or string of markers there. Each discreet unit is of a known length, allowing the user to expel only a single unit at each site. In a preferred embodiment, each discreet unit is of a fixed length, and a ratcheting or other metering device may be used at the distal end of the coaxial needle to extrude a fixed volume of needle content corresponding to a single discreet unit. In some embodiments, a stepper motor is used to aid in the precise expulsion of desired volumes of needle contents.

The contents of the needle may be loaded at either the tip end or the distal end. If loaded at the distal end, the distal end of the outer sheath optionally comprises a tapered or funnel shaped opening which facilitates the loading of markers, packing materials, and sealing materials into the lumen of the needle. These contents are then pushed to the tip end of the needle by inserting the stylet and advancing it towards the tip end of the needle.

The invention encompasses various kits comprising combinations of the novel elements described herein. In one embodiment, the invention comprises a kit comprising a marker and a plug of sealing material. In one embodiment, the invention comprises a 24 karat gold marker and a plug of Gelfoam™. In another embodiment, the invention comprises a marker, a plug of sealing material, and a coaxial needle. In another embodiment, the invention comprises a 24 karat gold marker, a plug of Gelfoam™ and a coaxial needle at least 30 cm in length.

Interstitial Brachytherapy

In an additional embodiment, the markers, delivery devices, and associated methods of the invention may be applied in interstitial brachytherapy, or internal radiotherapy. In interstitial brachytherapy, small amounts of radioactive material are placed within a tumor or cancerous region and slowly release radiation which treats the malignancy. Interstitial brachytherapy is used in the treatment of prostate, cervical, skin, breast, and other cancers. Typical brachytherapy employs small radioactive “seeds,” which are small rods comprising a radioactive material such as caesium-137, iodine-125, cobalt-60, iridium-192, and palladium 103. In some cases, the radioactive material is encased within a rod or capsule-like body, for example, made of titanium. In other cases, the brachytherapy implants comprise discreet bodies of radioactive material connected by biodegradable cord, resulting in “strands” of radioactive seeds that may be deployed around a target region.

The currently used brachytherapy implants are commonly smooth and do not employ anchoring moieties. Brachytherapy implants have been found to migrate from the site of insertion, for example as described in Reference 4 and Reference 5. Migration is dangerous to the patient, because the target area is not receiving therapeutic doses of radiation, while the migrating implants are irradiating healthy tissues. Accordingly, there is a need for preventing the migration of brachytherapy implants.

The migration of brachytherapy implants may be accomplished by the use of anchoring moieties. For example, an implant comprising a radiolucent body and one or more anchoring moieties may be used. Radioactive material is encased, encapsulated, or otherwise held within the radiolucent body, which allows delivery of radiation to the target site. The anchoring moieties are configured on the external portion of the body and aid in the anchoring of the body within the target tissue. Such an implant may further comprise a radio-opaque portion, for example gold, which allows visualization of the implant body. In an exemplary embodiment, the brachytherapy implant comprises a titanium capsule within which therapeutic radioisotopes and a radio-opaque marker are encased, the external body of the titanium capsule having one or more tooth-like projections of 0.05 to 0.5 mm in length.

Brachytherapy may also be improved by the use of sealing materials. For example, brachytherapy seeds may be needle injected into the target tissue with a following plug of Gelfoam™ or other swelling and/or hemostatic agent in order to physically seal and physiologically promote healing of the needle injection. The sealing material may comprise or be intermixed with compounds such as anti-infectives, healing promoters, therapeutic agents, radiosensitizing substances, chemotherapeutic agents, and other materials with ancillary functions beyond physically sealing the wound.

Methods of Using Markers

The markers, marker delivery devices, and associated methods of the invention may be applied in numerous medical and research contexts. They may be applied in any instance where the precise location of a target tissue, organ, or other region inside the body of an animal or person needs to be located by radio-imaging techniques with high accuracy. Exemplary uses include human medicine, veterinary medicine, and research.

The markers of the invention may be placed into any tissue or organ, for example, the bladder, prostate, liver, stomach, intestines, muscles, breasts, pancreas, bile ducts, brain, cervix, colon, esophagus, larynx, thyroid gland, uterus, subcutaneous fatty layers, or connective tissues. Additionally, the markers of the invention may be placed into tumors.

The markers of the invention may be visualized by any radio-imaging method known in the art, including, for example, MRI, CT, X-Ray, fluoroscopy, angiography, and ultrasound.

The devices and methods of the invention are especially amenable in external radiation therapy for the treatment of cancer. In such treatment regimens, the target region of the body is typically irradiated multiple times over a period of weeks. In such treatments, a high dose of irradiation is focused upon a specific region, e.g. a tumor or a site from which cancerous tissue has been removed and which may retain cancerous cells. The dual objectives in such treatment regimens are (1) to irradiate a specific region or regions, while (2) avoiding the unnecessary irradiation of surrounding healthy tissues, which may cause collateral damage to such tissues. In cases where the target area is composed of soft tissues, it is not possible to visualize the target area because the real-time imaging modalities available at the time of radiation therapy typically do not allow the visualization of soft tissues. In most cases, the types of radio-imaging which are integrated with radiotherapy delivery devices are conebeam CT and portal imaging, which do not resolve soft tissues. Accordingly, the area that is irradiated is often larger than the size of the target area, in order to increase the probability of hitting the target area with the radiation beam, which inherently results in over-irradiation of the patient. The devices and methods of the invention are especially useful for increasing the efficacy of such treatments, because the target region may be accurately located and radiation precisely delivered to it, while avoiding unnecessary irradiation of surrounding tissues.

For example, the devices and methods of the invention may be used in the treatment of prostate cancer. The use of fiduciary markers in the treatment of prostate cancer is previously known. Small gold fiduciary markers have been placed by needle into the apex and base of the prostate, allowing its precise location to be determined during radio-therapy and resulting in a more targeted treatment area, as reported in Reference 2. However, the fiduciary markers used in the prostate to date have been smooth, and they have been observed to migrate out of the prostate, as reported in Reference 3. Accordingly, the markers of the invention, comprising anchoring moieties, are highly resistant to migration and could be employed in the prostate. The markers of the invention, and other types of fiduciary markers, may also be placed into the prostate in conjunction with a plug of sealing material, as disclosed herein, improving the retention of such markers.

The devices and methods of the invention are especially amenable in the treatment of bladder cancer. In some cases, muscle-invasive bladder cancer is treated by removal of the tumor (resection), followed by radiation treatment (typically in conjunction with chemotherapy). The radiation treatment typically involves the irradiation of two target areas, a high-intensity irradiation of the tumor resection bed, and a low-intensity irradiation of the entire bladder. Prior to the commencement of treatment, the tumor resection bed is located by fluoroscopy and mapped to external landmarks on the body. Likewise, the location of the bladder is delineated by instillation of contrast agent solution and imaging with CT methods, and then mapped to external landmarks on the body. In subsequent radiation treatments, the external landmarks are used by the radiation oncologist to guide the high-field and low-field irradiation treatments, based on the predicted location of the tumor resection bed and the boundaries of the bladder, respectively.

Unfortunately, the bladder, being quite flexible and experiencing different stages of fullness throughout the day, is not static and changes shape and position at various times. Also, the bladder is adjacent to the digestive tract, which is in a constant state of movement. Accordingly, using external landmarks alone, the location of the resection bed and the boundaries of the bladder cannot be predicted with significant precision, resulting in the likely undertreatment of target areas and the overtreatment of collateral areas.

These shortcomings of the current treatment method may be overcome by various methods of the invention, as provided herein. Both the location of the tumor resection bed and the anatomical boundaries of the entire bladder may be advantageously delineated with great accuracy using the markers of the invention.

In one embodiment, the boundaries of the tumor resection bed are delineated by the use of one or more markers. Typically, the tumor resection bed comprises an area where the bladder mucosa layer has been mostly or completely removed, surrounded by healthy bladder mucosa. Markers of the invention may be placed into the bladder mucosa layer, or into the submucosal space between the bladder mucosa and bladder muscle layer, in the vicinity surrounding the tumor resection bed. Preferably, the markers are placed a distance from the edge of the resection bed, for example, about 1 cm. This is because necrotic bladder mucosa tissue at the margins of the resection bed may slough off, and markers placed too closely to this margin may be lost.

Any number of markers may be used to delineate the resection bed margins. In a preferred embodiment, 3 to 5 markers are utilized, oriented circumferentially around the margins. It is preferred that the markers be placed in a staggered orientation so that they will be distinguishable when viewed from either the anterior or posterior perspective, i.e. such that the markers will not overlap each other when viewed from certain angles. Because the shape and configuration of the bladder is in constant flux, the relative positions of the various markers may change over time when viewed on an imaging device. In order to accurately resolve the location of the tumor resection bed in all states of bladder fullness, in some cases it will be preferable to use heterogeneous markers. When heterogeneous markers are used, ambiguity as to the tumor resection bed location is reduced, because the different markers may be distinguished despite changes in their relative position caused by changes to the bladder shape and size. Heterogeneous marker shapes (e.g. curved vs. straight vs. spherical, etc.), sizes, and numbers (e.g. singlet vs. doublet vs. triplet, etc.) may be used at each of the marked locations.

The markers may be placed around the tumor resection bed using any method. A preferred method is the introduction of the markers by coaxial needle conjoined to a cystoscope. The combined cystoscope and coaxial needle are conjoined by any means, preferably one which allows them to move longitudinally along each other's axes, for example, within a lumen integrated into the body of a cystoscope, with rubber seals to prevent escape of liquid from the bladder.

The combined instrument is inserted through the urethra into the bladder, where the needle is then advanced beyond the tip of the cystoscope, for example extending 1 to 5 cm beyond the lens of the cystoscope, allowing the user to visualize, via the cystoscope, the tip of the needle as well as the interior features of the bladder. In a preferred embodiment, the end of the coaxial needle is curved, such that its sharpened tip is oriented transversally to the orientation of the cystoscope, for example at an angle of 45 to 90 degrees from the axis of the cystoscope. The user may then observe the interior of the bladder and locate the tumor resection bed by direct visualization.

The tip of the coaxial needle is then positioned in the location where the user wishes to deposit a marker. For example, viewing the tumor resection bed through the cystoscope, the user may position the marker one cm from the margin of the resection bed. The bladder mucosa layer is then gently pierced by advancing the coaxial needle into the tissue. Subsequently, a marker, and any associated sealing material, packing material, and other materials, are deposited into the tissue, or into the underlying submucosal space, by advancing the stylet of the coaxial needle enough distance to displace the marker and any other materials. Preferably, the coaxial needle is carefully advanced transversally into the bladder mucosa layer for a short distance before deposition of a marker, for example by 1 to 3 mm. This lateral tunneling into the bladder mucosa or submucosal space serves multiple purposes. First, it helps to insure that the marker is more reliably embedded into the bladder mucosa tissue. Secondly, it helps in orienting the long axis of the body marker parallel to the surface of the bladder, which aids in the retention of the marker in the bladder mucosa or submucosal space and prevents its implantation into the bladder muscle or surrounding tissue. Localization in the bladder mucosa is desired because the bladder muscle tissue and surrounding tissues may not precisely track the movement of the bladder wall (and the target resection bed). The marker deposition process is repeated at each location chosen to delineate the tumor resection bed. Subsequently, a radiographic image may be obtained to confirm the number and location of the markers, for example an anteroposterior X-ray image. If intra-operative radiographic images obtained immediately after marker placement suggest that the marker was placed outside of the intended target area (e.g. the placement was too deep, or not at the anatomic location desired), then another marker can be placed at the correct location, even if this is close to the initial location. This additional marker should preferably be distinguishable from the first marker, utilizing any of the unique designs described above (e.g. marker doublets or triplets, a curved marker, a marker with perpendicular hole(s), etc.)

During subsequent radiation treatments, the high-field radiation beam may be accurately targeted to the tumor resection bed by visualization of the markers. For example, the radiologist may employ fluoroscopic imaging of the bladder, allowing real-time monitoring of the resection bed area delineated by the marker or markers.

Additionally, the contours of the entire bladder may be located using the markers and devices of the invention. Markers may be placed into the bladder mucosa layer or the submucosal space around the margins of the bladder, delineating its entire shape, which such markers will be referred to as “anatomic markers.” For example, anatomic markers may be placed at locations delineating the normal geography of the bladder. Because the bladder is roughly spherical, it may be basically defined in a three-dimensional space using six markers. A first and second marker may delineate the extreme left and right margins of the bladder, as viewed from the anterior perspective. A third marker placed in the most the most cephalad margin and a fourth marker placed at the most caudal margin (again, as viewed from the anterior perspective) define the head-to-foot axis of the sphere. A fifth marker placed at the bladder dome and a sixth marker placed at the posterior margin define an up-down axis, as viewed from the lateral perspective. In practice, it is not always necessary to define all six points, and subsets of points may yield adequate information for fairly precise targeting of the bladder boundaries. For example, the cephalad margin of the bladder may be well approximated by bony landmarks. For example, knowing the location of the left and right margins (as viewed from the anterior perspective) and the posterior margin will provide enough information to define the lower hemisphere of the bladder, which is adjacent to the essential collateral tissues of the digestive system and other organs. When placing markers to define the margins of the bladder (for example from the anterior or lateral perspective), it is important that the markers be placed in the extreme margins, to ensure that the entire bladder lies within the area defined by the markers, and thus allows for more accurate targeting of the whole bladder in radiation therapy. Alternatively, markers can also be used to delineate abnormal bladder anatomy, such as, for example, the location and margins of a bladder diverticulum, or, the location of previous (and now healed, and visibly disease-free) tumor resection sites (which can harbor occult tumor disease) so that these can be targeted for high-dose radiation.

Placement of the anatomical markers delineating the borders of the bladder may be accomplished by any means. In a preferred embodiment, a conjoined cystoscope/coaxial needle combination, as described above, is utilized in parallel with whole bladder imaging. Whole bladder imaging is accomplished by instilling the bladder with dilute contrast agent. Dilute contrast agent comprises a solution that is sufficiently transparent such that bladder features may be visualized by use of the cystoscope, but which is sufficiently radio-opaque to allow resolution of the bladder margins by a real-time imaging modality such as fluoroscopy, conebeam CT, etc. For example, 25-50% strength contrast agent solution may be used. For example, one part Conray™43 (Iothalamate Meglumine Injection U.S.P. 43%) (by Mallinckrodt Inc.) may be diluted with 4-5 parts saline. Such a solution, instilled in the bladder, will delineate the bladder as a light gray mass when imaged by fluoroscopy, while allowing simultaneous cytoscopic visualization of bladder features. Preferably, the tip of the co-axial needle is radio-opaque, or has been adorned or coated with radio-opaque materials, such that it may be visualized by real-time imaging, aiding the user in precise placement of the anatomic markers along the margins of the bladder.

During a radiation treatment session, the radiologist is unable to image the soft tissues of the bladder directly, but may image the anatomic markers, for example by fluoroscopy, conebeam CT, or portal imaging, and may then direct low-field radiation to the area delineated by the markers, increasing accuracy of whole-bladder irradiation and reducing the deleterious irradiation of surrounding tissues and organs.

Anatomic marking of the bladder margins, as described above, provides a method of delineating the bladder that is vastly more accurate than current methods of mapping one-time bladder locations to external and bony landmarks. However, the shape, size, and location of the bladder will vary over time as the bladder is filled and emptied of urine, and such changes may not be fully resolved by the use of anatomic markers alone. In those situations where even greater precision is desired, anatomic bladder markers may be used to generate a bladder boundary predictive tool (“BBPT”) for precise targeting of the whole bladder in any state of fullness.

The BBPT is generated using simultaneous imaging of the whole bladder and anatomic markers, as follows. First, the bladder is instilled with a small amount of dilute contrast agent solution, of volume equal to what the particular patient could be expected to have within their bladder on each day of treatment. The dilute contrast agent solution must be sufficiently concentrated that the visualization of the whole bladder may be observed by a selected imaging modality, for example by fluoroscopy, while being sufficiently transparent for visualization of anatomic markers that have been placed in the bladder. For example, such a solution may be made using one part Conray™43 (Iothalamate Meglumine Injection U.S.P. 43%) (by Mallinckrodt Inc.) diluted with 4-5 parts saline.

Next, the bladder anatomic markers are placed at various positions in the bladder, as described above. The contrast within the bladder allows the urologist placing the markers to identify the exact locations for anatomic marker placement. Next, once the anatomic markers have been placed, the dilute contrast agent is instilled into the bladder in varying increments, ranging from empty to full (e.g., 240 ml, which is full for the average adult bladder), for example, the bladder may be instilled with zero ml, 60 ml, 120 ml, 180 ml, and 240 ml of dilute contrast agent solution, and then may be serially emptied by release of aliquots of contrast solution. When this filling/emptying process is performed, the size and the shape of the bladder (as visualized by the mass of contrast agent, which delineates the inner wall boundaries of the bladder) is observed to change significantly, in a consistent fashion, as the bladder is filled and emptied. Simultaneously, the markers placed in and around the bladder are observed to move, relative to each other, in a consistent fashion. Thus, the distance between any two markers, and the angle between any two markers (relative to a fixed vector such as the patient's left-right axis), is correlated with the size and shape of the bladder at any given state of fullness. Accordingly, this correlation (bladder volume, distance between two or more markers, and bladder contour, defined by the contrast-fluid/bladder wall interface) can be used to predict the location of bladder contours using marker positions relative to each other.

In order to generate a BBPT for an individual patient, bladder marking and the filling/emptying process, as described above, is performed. At each increment of fullness, a radiographic image is captured which simultaneously visualizes both the anatomical boundaries of the bladder and the location of anatomic markers. If markers delineating the tumor resection bed have also been placed into the bladder, these will be imaged as well. The images are subsequently analyzed to determine the mathematical relationship between the location of each marker relative to each other marker and the corresponding location of the margins of the bladder. The BBPT comprises the correlations derived between relative marker positions and the anatomic limits of the bladder, and allows imaging of the markers alone to be used for prediction of the position of the bladder margins, in any state of bladder fullness.

Once the correlations between relative marker locations and bladder contours have been established for an individual patient, the BBPT may be applied in guiding adjustments to the radiation field-area and dosage, on each day of radiation treatment. For example, at the time of each radiation therapy session, the radiologist may observe the position of the markers using an imaging modality, such as, for example portal imaging, or cone-beam CT. Using the previously derived correlations between marker position and the location of bladder boundaries, positional information about the markers is used to predict the current location of the bladder's boundaries. The whole-bladder radiation dose is then targeted appropriately and, can be adjusted depending on the state of bladder fullness.

BBPT's established for individual patients may be compiled to create a “universal BBPT” for use by carefully selected patients. With a uniform and consistent system of marking bladder features across patients that share common features (e.g. age, similar bladder capacities, absence of bladder anatomic abnormalities, etc.), and an adequate sample size, a nonogram representative of the average patient may be derived. Subsequently, this nonogram may be applied to new patients by analysis of their anatomic markers, obviating the need for the bladder filling and calibration step.

The invention encompasses software programs for the generation of the BBPT. Such programs may include user interfaces (e.g. mouse, touchscreen, pen, etc.) that allow the user to manually delineated individual marker positions and bladder boundaries in each image. Alternatively, individual marker positions and bladder boundaries in each image may be delineated using any number of image analysis software tools known in the art, e.g. pattern recognition algorithms tailored to detect the appearance of markers and bladder margins in the images. Once marker positions and bladder contours have been delineated, linear regressions and other statistical analysis methods may be applied to derive correlations between marker positions and bladder boundaries.

BBPT software programs may be utilized in real time to guide radiation therapy. For example, real-time imaging and analysis of anatomic marker positions may be used to generate a predicted map of the bladder, which such map may be automatically interfaced with radiation delivery equipment. For example, a visual representation of the predicted bladder boundaries may be superimposed on fluoroscopy, portal imaging, or cone-beam CT images of the patient, guiding the radiation therapist to apply the whole-bladder dose in the appropriate area.

### EXAMPLES

### Example 1

### Construction of Fiduciary Markers with Anchoring Moieties and Delivery Needle

The starting material for the fiduciary markers was 24 karat gold wire (W.E. Mowrey Company). Large markers (with a body size of approximately 3.2 mm long by 1.1 mm wide) were constructed by cutting 3.2 mm lengths of 17 AWG wire. Small markers (with a body size of approximately 2.1 mm long by 0.65 mm wide) were constructed by cutting 2.1 mm lengths of 22 AWG wire.

On each marker, four parallel rows of anchoring moieties were made along the long axis of the body, each row spaced at 90 degree intervals circumferentially around the circular body of the marker.

Anchoring moieties were made by cutting a short distance into the body of the marker with a sharp surgical scalpel at an angle of 30-45 degrees. After cutting into the wire, a slight rotation of the scalpel towards the perpendicular caused the flaps to flare out away from the body of the wire. The resulting flaps of lifted material resembled spines or triangular teeth and they projected from the body of the marker at a distance ranging from 0.05 to 0.3 mm. Approximately 2-3 anchoring moieties per mm of marker body were made in each row.

Viewing the long axis of the marker in a horizontal orientation, within each row of anchoring moieties, the anchoring moieties on the left and right halves were of opposite orientation, with their pointed ends facing back towards the midline at an approximate angle of 30 degrees. A schematic representation of the markers and the anchoring moieties is depicted in FIG. 1.

For delivery of the large markers, a customized 30 cm long 16 AWG diameter surgical grade stainless steel coaxial needle was constructed (Popper and Sons, Inc., NY, USA). For delivery of the small markers, a customized 30 cm long 18 AWG diameter surgical grade stainless steel coaxial needle was constructed (Popper and Sons, Inc., NY, USA). The end of each needle was beveled at an approximate angle of 35-40 degrees.

Each coaxial needle contained a surgical grade stainless steel internal stylet, having a distal end that extended from the distal end of the outer needle sheath and which fitted with a circular plastic thumb plate, approximately 8 mm in diameter. The distal end of the outer housing was circumscribed by a metal lip, allowing the end of the needle to be gripped with the index and middle fingers and the stylet to be advanced by depressing the thumb plate.

Markers were loaded into the distal end of the needle. In some cases, the diameter of the tined markers was larger than the diameter of the needle lumen, and gently pushing the markers into the lumen bent the anchoring moieties downward slightly, allowing the markers to move through the lumen. Immediately distal to each marker, a small plug of morcellized Gelfoam™ (Pfizer), was loaded. The Gelfoam plugs were made by wetting Gelfoam material, masticating it with tweezers, and packing the resulting mass into the lumen of the needle. The stylet was then inserted and advanced until the marker and following Gelfoam plug were located just below the opening of the needle tip.

### Example 2

### Use of Fiduciary Markers with Anchoring Moieties to Delineate Tumor Resection Beds

Between January 2007 and March 2011, the gold fiduciary markers described in Example 1 were tested in a total of 10 patients. Each patient had been diagnosed with a single bladder tumor (focal, T2, NX, M0 transitional cell bladder cancer) and had undergone a primary endoscopic resection of a single tumor from the bladder wall. The patient pool consisted of seven men and three women, mean age 77.4 and 74.7 years, respectively.

Fiduciary markers were implanted at the time of bladder tumor repeat resection/fulguration. Using the coaxial delivery needle described in Example 1, conjoined to a standard cystoscope, 3-5 markers were implanted in each patient in a roughly circular pattern outlining the tumor resection bed, for a total of thirty-nine markers placed. Markers were placed in a staggered orientation, such that all markers were visible from both anterior-posterior and lateral radiographic views. Markers were placed at an approximate distance of 1 cm from the edge of the tumor resection bed. Fourteen of the 39 markers (36%) placed were of the small size (body dimensions of 2.1 mm×0.65 mm), and twenty five of the thirty nine markers (64%) were of the large size (body dimensions of 3.2 mm×1.1 mm).

Markers were placed by burrowing the tip of the coaxial needle approximately 2 mm into the healthy bladder mucosa layer at and angle of 30-40 degrees and depressing the stylet by a distance of 2-3 mm. Each marker was co-injected with a following Gelfoam™ plug, as described in Example 1, which such plug was intended to aid in sealing the needle puncture wound and preventing escape of the marker.

For each of two patients, one marker fell-out during placement (as a result of the placement needle not being advanced sufficiently into the sub-mucosal space). On both instances, the marker was retrieved with an endoscopic grasping instrument, and placed correctly as described.

A schematic drawing of the bladder was made by the surgeon at the time of marker placement to mark the site of the tumor resection area and location of all markers. These notes were referenced by the treating radiation oncologist. All patients received oral prophylactic antibiotic for 5 days post-operatively. After marker placement, all patients underwent pelvis KUB X-Ray imaging post-operatively to confirm placement and location of all markers.

All (100%) markers were clearly visible on KUB, CT scan, and MV/conebeam portal imaging. Marker fall-out rate before commencement of radiotherapy was 0%. Quantitative review of all portal images suggested no evidence of marker migration to within +1 mm. No intraoperative or post-operative complications occurred. Electrocautery was used only once to control mucosal bleeding

During the course of the study, eight of the ten patients underwent and completed radiation therapy. Among these eight patients, a total of 32 markers had been placed. Only one patient experienced marker loss, during the last quarter of radiotherapy, the marker having fallen out and being passed in the urine. Thirty-one of thirty two markers (97%) remained in place during the course of radiation therapy.

All patients who received radiotherapy underwent CT scan imaging for dosimetry planning, and portal imaging at time of daily radiotherapy. All patients were treated with IMRT using 6MV photons. Patients were aligned with one of two techniques. Either daily AP and lateral-view plain portal images were taken and the gold markers were aligned on the reference digitally recreated radiograph, or, daily MV cone-beam CT was performed and aligned to the gold markers on the planning CT images.

In the radiation therapy regimen performed on these patients, gross tumor volume (GTV) was targeted for high dose radiation. In typical patients undergoing this radiation therapy regimen at the same institution, due to the inability to visualize the bladder wall tumor resection site on portal imaging during daily treatment, the smallest portion of the bladder that is targeted for GTV is about 25% of the total bladder. In those patients having the fiduciary markers, the area of the bladder targeted for high-dose radiation was about 4-6% of bladder area, representing a 75-83% reduction in the high radiation treatment area.

This study demonstrates that gold fiduciary markers with anchoring moieties, injected in conjunction with a sealing material, may be easily delivered into bladder submucosa endoscopically, to outline the location of a muscle invasive bladder or location in real time during daily radiotherapy treatment. The markers were retained at a very high rate (>90%) during the course of treatment. The markers allowed accurate locating of the tumor resection site and resulted in a very significantly reduced size of the high-radiation treatment area.

### Example 3

### Delineation of the Whole Bladder Using Fiduciary Markers

In this Example, the use of the fiduciary markers to delineate the boundaries of the whole bladder is demonstrated. This study was conducted in a male subject having been diagnosed with a 2-3 cm muscle invasive bladder tumor located at the dome of the bladder. The tumor was removed by transurethral resection.

Three and a half months later, fiduciary markers were implanted in the patient to delineate the tumor resection bed, using the same general method as described in Example 2. The markers had body dimensions of 2.6 mm long by 0.7 mm wide, having four rows of anchoring moieties, made and configured as described in Example 1. Markers were co-injected with following Gelfoam™ plugs, as described in Example 1.

Four sites were marked with fiduciary markers. A single marker was placed at the right lateral limit of tumor area. A single marker was placed at the left lateral limit of tumor area. A single marker was placed at the superior-midline tumor margin (located on the dome of the bladder). At the inferior midline margin of tumor area, two markers were co-injected side-by-side, creating a distinguishable doublet in order to distinguish this site from the single marker placed at the superior margin.

Additionally, two anatomic markers were placed, one at the left lateral bladder wall margin and one at the right lateral bladder wall margin. Anatomic markers were placed after instilling the bladder with 60 ml of dilute contrast agent solution (1 part Conray™ 43 diluted with 4-5 parts saline). Fluoroscopic imaging was used to observe the lateral margins of the bladder to guide the coaxial needle to these margins for placement of a marker at each.

Anatomic marking of the bladder dome was not necessary in this case because one of the tumor resection bed markers was already located at the dome.

Following implantation of the markers, the bladder was filled with 240 ml (full) of dilute contrast solution in 60 ml increments. At each stage of bladder fullness (0 ml, 60 ml, 120 ml, 180 ml, and 240 ml), fluoroscopic images were captured. In each image, the borders of the bladder are clearly delineated (appearing light gray in the image) and the fiduciary markers are clearly visible (appearing black).

The relative locations of the markers to each other was found to change at each stage of bladder fullness. For example, the marker located at the superior aspect of tumor is also located in the “dome” (or anterior wall when patient is supine). At low bladder volume, where the “dome” drops anteriorly, this marker appears inferiorly located. Likewise the shape and size of the bladder as a whole was observed to change dramatically during the filling and emptying process. This same anatomic marking process has been repeated with four additional patients.

### Example 4

### Correlating Relative Marker Position to Changes in the Bladder Boundaries

Using the methods described in Example 2, three tined fiduciary markers were implanted in the bladder mucosa of a patient. The markers were 2.1 mm in length and 0.65 mm in width, with four rows of tines, arranged at 90-degree orientation from each other circumferentially around the axis of the marker, with about 2-3 tines per mm of marker. The tines were angled 30-40 degrees from the body of the marker, with those on the left and right halves oriented at opposing angles.

One marker was placed in to the bladder mucosa at the lateral-most aspect of the right lateral wall (Marker #1). The second marker was placed at the junction of the posterior wall and dome (Marker #2). The third marker was placed at the intersection of the bladder midline and at the right posterior-lateral wall (Marker #3). Viewing the markers from the anterior perspective, Marker #1 was placed at 12 o'clock on the circular mass defining the bladder, Marker #2 was placed at about 10:30 o'clock, and Marker #3 was placed at 9 o'clock.

The markers were placed with the bladder partially full, containing 60 ml. diluted contrast agent (as described in Example 3). Following marker placement, using the methodology described in Example 3, the absolute location of each marker was assessed, and its location relative to the bladder mucosa, (using anterior-posterior view fluoroscopic radiographs) relative to varying states of bladder fullness with diluted radiographic contrast solution. The bladder was serially filled by instilling 60 ml aliquots of the dilute contrast agent solution, being filled with 0, 60, 120, 180, and 240 ml, and then serially emptied by draining 60 ml aliquots of contrast agent solution. At each stage of filling and emptying, a fluoroscopic image was captured, from the anterior perspective.

In each fluoroscopic image, the contours of the inner bladder wall were clearly visualized by the mass of contrast agent solution. The tined fiduciary markers placed within the bladder mucosa were also clearly visible. In the series of images captured as the bladder was filled, the position of the bladder margins changes substantially at each stage of fullness, as the bladder grew in size and changed shape. As the bladder was emptied, its boundaries returned to their original positions.

In each image, various inter-marker distances and angles were measured, including the distance between Marker #1 and Marker #2, the distance between Marker #1 and Marker #3, the distance between Marker #2 and Marker #3, and the angle between Marker #1 and Marker #2 (relative to the vertical axis of the image). As set forth in Table 1, at each stage of bladder fullness, the inter-marker distances and the angle between Marker #1 and Marker #2 changed in a consistent fashion. The results demonstrate that inter-marker distances and angles change in a predictable fashion as the position of the bladder boundaries change.

TABLE 1

| | Bladder Filling Volume (ml) | | | | |
|---|---|---|---|---|---|
| | 0 | 60 | 120 | 180 | 240 |
| Distance: marker#1 to | 0.49 | 0.53 | 0.61 | 0.69 | 0.79 |
| #2 (inches) | | | | | |
| Distance: Marker #2 to | 0.49 | 0.83 | 1.02 | 1.13 | 1.25 |
| #3 (inches) | | | | | |
| Distance: Marker #1 to | 0.97 | 1.3 | 1.54 | 1.72 | 1.95 |
| #3 (inches) | | | | | |
| Angle between # 1 & #2 | 0 | 26 | 19 | 26 | 29 |
| (degrees) | | | | | |

### Example 5

### The Effect of Anchoring Moieties on Marker Mobility within Animal Tissues

The tension required to pull fiduciary markers with and without micro-tine anchoring moieties through various tissue types was measured with a digital tension-meter (Digital Force Gauge Model EG2 (Mark 10 Instruments)).

Twelve 24-K gold fiduciary markers, 3 mm long by 0.9 mm wide were made. Six of the markers had four rows of micro-tine anchoring moiety teeth, projecting at a 20-40 degree angle from the body of the marker, spaced at approximately 2-3 micro-tines per mm, made as described in Example 1 except that all tines were pointed in one direction. The remaining six markers did not have any anchoring moieties. Each marker was soldered to 24-K gold wire of slightly smaller diameter than the markers, using gold soldering agent.

The end of the wire opposite to where the marker was soldered was bent into a ring-shape. A string was tied to the ring on each gold “marker-wire.” The other end of the string was tied to the hook of the digital tension meter.

Different fresh meat tissues were bisected with a sharp knife. The meat tissues were beef flank-steak, beef liver, pork intestine, and chicken liver. In addition, fluffy cotton was used. The “marker-wire” was placed between the two halves of the meat tissue or between two cotton wads, and then a 4-oz metal weight was placed on top of the marker-wire sandwiched between two layers of meat. The marker-wire was then pulled from between the tissues, at a slow rate (2 inches per second). The peak tension force that occurred during pulling was recorded. Using six different tined and untined markers, total of 20 trials were repeated for each material.

The mean maximum tension recorded for movement of the untined marker wires through the various types of meat ranged from 0.007 lbs (chicken liver) to 0.16 lbs (beef flank steak). For the tined marker-wires, the mean maximum tension recorded for movement of the untined markers through the various types of meat ranged from 0.0305 lbs (chicken liver) to 0.0387 lbs (beef flank steak). For movement through cotton, the mean maximum tension recorded for the untined marker-wires was 0.00975 and for the tined marker-wires was 0.1838.

For all materials, the mean maximum tension recorded was higher for tined markers than for untined markers. The ratio of maximum force recorded for tined marker-wires relative to untined marker-wires was 2.38 in beef flank steak, 2.79 in beef liver, 2.55 in pork intestine, 4.36 in chicken liver, and 18.85 in cotton.

In summary, these results demonstrate that the movement of tined markers in animal tissues requires more than 2-4 times greater force than the movement of untined markers, demonstrating the utility of anchoring moieties in resisting movement of the marker through animal tissues.

Unless otherwise defined, all technical and scientific terms used herein have the same meaning as commonly understood by one of ordinary skill in the art to which this invention belongs. The materials, methods, and examples included herein are illustrative only and not intended to be limiting. Other features, objects, and advantages of the invention will be apparent from the detailed description, and from the claims. All publications, patent applications, patents, and other references mentioned herein are incorporated by reference in their entirety.

1-34. (canceled)

35. A device for implanting one or more objects within the tissue of an animal, comprising
a needle;
one or more objects residing within the lumen of the needle, the one or more objects substantially not comprising a material which swells when wetted;
a plug of sealing material residing within the lumen of the needle, the plug of sealing material being located distally to the one or more objects within the lumen, with respect to the tip of the needle.

36. The device of claim 35, wherein
the one or more objects comprises a radio-opaque tissue marker.

37. The device of claim 36, wherein the radio-opaque tissue marker comprises
a material selected from the group consisting of
gold, silver, palladium, and platinum.

38. The device of claim 36, wherein the radio-opaque tissue marker comprises
one or more anchoring moieties.

39. The device of claim 35, wherein the plug of sealing material comprises
a material selected from the group consisting of
porcine skin gelatin sponge material, polyvinyl alcohol sponge material, oxidized regenerated cellulose material, collagen sponge material, poly(ethylene oxide), and a hydrogel.

40. The device of claim 35, wherein the plug of sealing material comprises
a substantially cylindrical shape.

41. The device of claim 40, wherein
the diameter of the plug of sealing material is within the range of 75 to 99 percent of the diameter of the lumen.

42. The device of claim 40, wherein
the length of the plug of sealing material is in the range of 10 to 200 percent of the length of the one or more objects in the lumen, length being measured along the long axis of the lumen.

43. The device of claim 36, wherein
a series of at least two units is present, the units being arranged end-to-end within the lumen of the needle and each unit comprising at least one radio-opaque tissue marker and a sealing plug, the sealing plug being located distally to the at least one radio-opaque body within the lumen, with respect to the tip of the needle.

44. The device of claim 43, further comprising
a ratcheting or metering means which allows a single unit to be discreetly dispensed from the tip of the needle.

45. The device of claim 36, wherein
the one or more objects comprises a doublet of two radio-opaque tissue markers and/or a triplet of three radio-opaque tissue markers.

46. The device of claim 35, further comprising
a cystoscope, conjoined with the needle such that the tip of the needle is visible within the field-of-view of the cystoscope.

47. The device of claim 46, wherein
the tip portion of the needle is curved, such that the tip of the needle is oriented transversally to the axis of the field-of-view of the cystoscope.

48. The device of claim 35, wherein
a portion of the tip of the needle is radio-opaque.

49. The device of claim 35, wherein the one or more objects comprises
a brachytherapy implant.

50. A method of implanting one or more objects within the tissue of an animal, the one or more objects substantially not comprising a material that swells when wetted, comprising
injecting the one or more objects into the tissue of the animal with a needle, wherein a plug of sealing material is co-injected behind the one or more objects, the plug of sealing material comprising a material which swells when wetted.

51. The method of claim 50, wherein
the plug of sealing material comprises a material selected from the group consisting of
porcine skin gelatin sponge material, polyvinyl alcohol sponge material, oxidized regenerated cellulose material, collagen sponge material, poly(ethylene oxide), and a hydrogel.

52. The method of claim 50, wherein
the one or more objects comprises a radio-opaque tissue marker.

53. The method of claim 52, wherein
the radio-opaque tissue marker comprises a gold body with at least four anchoring moieties.

54. The method of claim 50, wherein
the tissue is tissue of the bladder.
